(19) United States
(12) Patent Application Publication
(10) Pub. No.: US 2025/0134356 A1
(43) Pub. Date: May 1, 2025

(54) CONNECTOR FOR A FLEXIBLE ELONGATE MEMBER INSERTABLE INTO A BODY

(71) Applicant: BOSTON SCIENTIFIC SCIMED, INC., Maple Grove, MN (US)

(72) Inventors: Jennifer Lewitzky, Lexington, MA (US); Colby Harris, Norfolk, MA (US); Austin Grant Johnson, Worcester, MA (US)

(73) Assignee: BOSTON SCIENTIFIC SCIMED, INC., Maple Grove, MN (US)

(21) Appl. No.: 18/925,786

(22) Filed: Oct. 24, 2024

Related U.S. Application Data
(60) Provisional application No. 63/593,109, filed on Oct. 25, 2023.

(51) Int. Cl. A61B 1/00; A61B 17/00

(52) U.S. Cl. CPC A61B 1/00128; A61B 2017/00477

(57) ABSTRACT

Devices, systems, and methods for coupling an elongate member with a shaft of an endoscope. The device for coupling an elongate member with a shaft of an endoscope may include a first contoured portion configured to mate with an outer surface of the shaft and a second contoured portion configured to couple with the elongate member. When the first contoured portion is mated with the outer surface of the shaft and the second contoured portion is coupled with the elongate member, the elongate member may be secured relative to the shaft. When secured relative to the shaft, the elongate member may be able to move longitudinally relative to the shaft.

### CROSS-REFERENCE TO RELATED APPLICATIONS

This application claims the benefit of priority under 35 U.S.C. § 119 to U.S. Provisional Application No. 63/593,109, filed Oct. 25, 2023, the entire disclosure of which is hereby incorporated by reference herein for all purposes.

### TECHNICAL FIELD

This disclosure relates generally to connector assemblies and methods, and particularly to connectors for coupling elongate members with an endoscope.

### BACKGROUND

A wide variety of intracorporeal medical devices and systems have been developed for medical use, for example, for transluminal procedures. Some of these devices and systems include guidewires, catheters, catheter systems, transluminal instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In a first example, a device for coupling an elongate member with a shaft of an endoscope may comprise a first contoured portion configured to mate with an outer surface of the shaft of the endoscope and a second contoured portion configured to couple with the elongate member, and wherein when the first contoured portion is mated with the outer surface of the shaft and the second contoured portion is coupled with the elongate member, the elongate member may be secured relative to the shaft and may adjustable relative to the shaft.

Alternatively or additionally to any of the examples above, the second contoured portion may be configured such that the elongate member is able to adjust rotationally and longitudinally relative to the shaft when the elongate member is coupled with the second contoured portion and the first contoured portion is mated with the outer surface of the shaft.

Alternatively or additionally to any of the examples above, the second contoured portion may be part of a coupling component configured to couple with the elongate member via a snap connection.

Alternatively or additionally to any of the examples above, the first contoured portion may be part of a coupling component configured to couple with the shaft via a snap connection.

Alternatively or additionally to any of the examples above, the first contoured portion and the second contoured portion may be monolithic.

Alternatively or additionally to any of the examples above, the device may further comprise a fixture defining the first contoured portion and the second contoured portion and a band configured to engage the fixture and couple the fixture to the shaft of the endoscope.

Alternatively or additionally to any of the examples above, the band may be configured to extend around the shaft and an outer surface of the fixture, the outer surface of the fixture faces a direction that is facing away from a surface of the first contoured portion.

Alternatively or additionally to any of the examples above, the band may have a first end secured to the fixture and a second end secured to the fixture.

Alternatively or additionally to any of the examples above, the device may further comprise a band defining the first contoured portion and a fixture configured to secure the band relative to the shaft of the endoscope, wherein the fixture defines the second contoured portion and a compartment configured to receive the band.

Alternatively or additionally to any of the examples above, the fixture may include a first sub-component and a second sub-component configured to couple with the first sub-component to define the compartment.

Alternatively or additionally to any of the examples above, the device may further comprise a band defining the first contoured portion and a fixture comprising an opening configured to receive the band, and wherein the opening may be located at one of a bottom of the fixture configured to face the shaft of the endoscope and a side of the fixture configured to face a direction parallel to a longitudinal axis of the shaft when the band is received within the fixture and the first contoured portion is mated with the shaft.

In a further example, a system for coupling an elongate member with a shaft of an endoscope may comprise a first coupling component and a second coupling component configured to couple with the elongate member, and wherein the first coupling component may be adjustable relative to the second coupling component to releasably couple with the shaft of the endoscope and when the first coupling component is releasably coupled with the shaft and the second coupling component is coupled with the elongate member, the elongate member may be secured relative to the endoscope and may be able to move longitudinally relative to the shaft.

Alternatively or additionally to any of the examples above, the first coupling component is annular and flexible, and the system may further comprise a delivery tool configured to expand the first coupling component to a diameter greater than a diameter of the shaft for delivery of the first coupling component and the second coupling component to a position along a length of the shaft.

Alternatively or additionally to any of the examples above, the second coupling component may have a groove in an outer surface, the groove is configured to receive the first coupling component.

Alternatively or additionally to any of the examples above, the delivery tool may have a groove in an outer surface, the groove is configured to receive the first coupling component.

Alternatively or additionally to any of the examples above, the first coupling component is a portion of a clip and the second coupling component is a portion of the clip.

In a further example, a method of coupling an elongate member to a shaft of an endoscope may comprise coupling a first coupling component to the shaft of the endoscope, wherein the elongate member is coupled to a second coupling component and coupling the first coupling component to the shaft secures the elongate member relative to the shaft at an axial location between a first end of the shaft and a second end of the shaft, and wherein the elongate member may be able to move longitudinally relative to the second coupling component when coupled with the second coupling component and the first coupling component is coupled with the shaft.

Alternatively or additionally to any of the examples above, the method may further comprise coupling the second coupling component with the elongate member, and wherein coupling the second coupling component with the elongate member includes snapping the elongate member into the second coupling component.

Alternatively or additionally to any of the examples above, the first coupling component may be a portion of a clip and the second coupling component may be a portion of the clip.

Alternatively or additionally to any of the examples above, the first coupling component comprises a band and coupling the first coupling component to the shaft comprises positioning the band around the shaft.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of a distal end of an illustrative endoscope shaft with an elongate member extending along the shaft;

FIG. 3. depicts a schematic view of an illustrative connector extending around an endoscope shaft and an elongate member;

FIG. 4 depicts a schematic perspective view of an illustrative connector configured to couple an elongate member with an endoscope shaft;

FIG. 5 depicts a schematic end view of the illustrative connector depicted in FIG. 4;

FIGS. 6A-6C depict a schematic technique for coupling the elongate member with the endoscope shaft using the connector depicted in FIG. 4;

FIG. 7 depicts a schematic perspective view of the elongate member coupled with an endoscope shaft using the illustrative connector depicted in FIG. 4;

FIG. 8 depicts a schematic perspective view of an illustrative connector configured to couple an elongate member with an endoscope shaft;

FIG. 9 depicts a schematic end view of the illustrative connector depicted in FIG. 8, with the band in a coupled configuration;

FIG. 10 depicts a schematic perspective view of an illustrative connector, with a band in an uncoupled configuration;

FIG. 11 depicts a schematic perspective view of the elongate member coupled with an endoscope shaft using the illustrative connector depicted in FIG. 10;

FIG. 12 depicts a schematic perspective view of an illustrative connector configured to couple an elongate member with an endoscope shaft;

FIGS. 13A-13D depict a schematic technique for coupling the elongate member with the endoscope shaft using the connector depicted in FIG. 12;

FIG. 14 depicts a schematic perspective view of an illustrative connector configured to couple an elongate member with an endoscope shaft;

FIG. 15 depicts a schematic cross-sectional view of a portion of the illustrative connector depicted in FIG. 14, taken along line 15-15;

FIG. 16 depicts a schematic cross-sectional view of a portion of the illustrative connector depicted in FIG. 14, taken along line 16-16;

FIG. 17 depicts a schematic perspective view of an illustrative connector configured to couple an elongate member with an endoscope shaft;

FIG. 18 depicts a schematic perspective view of a portion of the illustrative connector depicted in FIG. 19;

FIG. 19 depicts a schematic cross-sectional view of the portion of the illustrative connector depicted in FIG. 18, taken along line 19-19;

FIG. 20 depicts a schematic perspective view of an illustrative connector configured to couple an elongate member with an endoscope shaft; and

FIGS. 21A and 21B depict a schematic view of an illustrative technique for connecting portions of the connector depicted in FIG. 20.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in transluminal medical procedures. However, it should be noted that reference to this particular procedure is provided only for convenience and not intended to limit the disclosure. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s).

Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “a configuration”, “some configurations”, “other configurations”, etc., indicate that the configuration(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with a configuration, the particular feature, structure, or characteristic may be implemented in connection with other configurations, whether or not explicitly described, unless clearly stated to the contrary. That is, the various individual elements described below, even if not explicitly shown in a particular combination, are nevertheless contemplated as being combinable or arrangeable with each other to form other additional configurations or to complement and/or enrich the described configuration(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

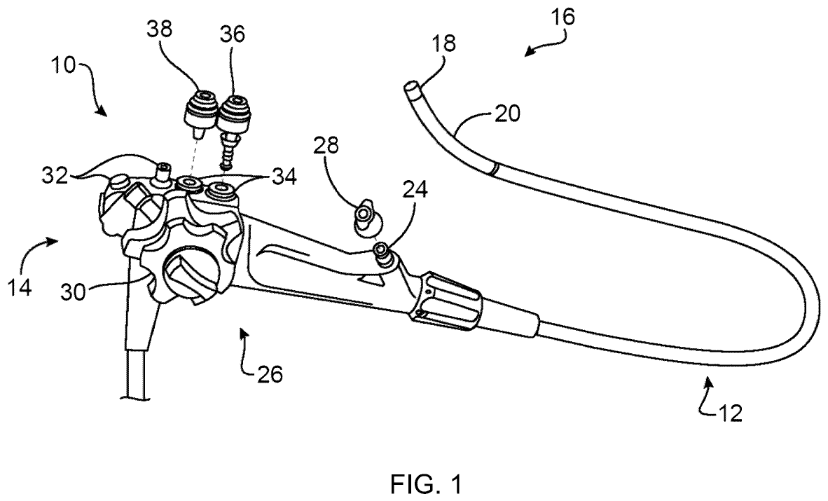

With reference to FIG. 1, an illustrative endoscope 10 is depicted in a schematic perspective view. The endoscope 10 may include an elongate tube or shaft 12 that is configured to be inserted into a subject (e.g., a patient).

The endoscope 10 may include a proximal portion 14 and a distal portion 16. The distal portion 16 of the endoscope 10 may house an imager (e.g., CCD or CMOS imager) (not shown). In some cases, a light source of an endoscope system including the endoscope 10 may feed illumination light to a distal portion 16 of the endoscope 10.

The endoscope shaft 12 may include a distal tip 18 (e.g., a distal tip unit adapted to be inserted into a body cavity of a patient) provided at the distal portion 16 of the shaft 12 and a flexible bending portion 20 proximal of a distal tip 18. The flexible bending portion 20 may include an articulation joint (not shown) to assist with steering the distal tip 18, but this is not required.

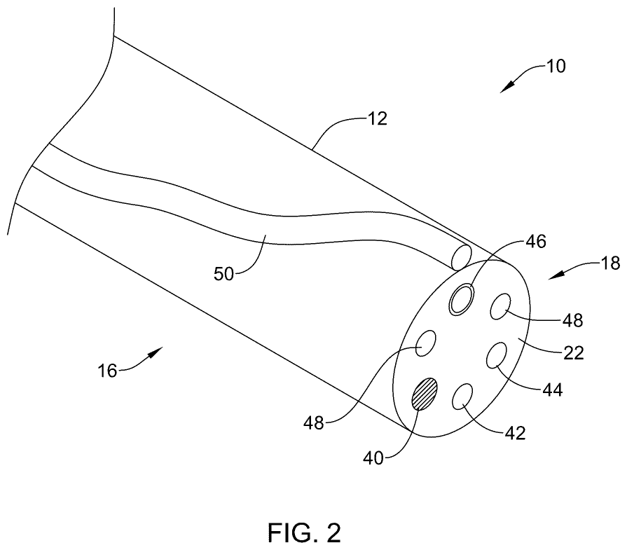

The distal tip 18 of the endoscope 10 may include an end face (e.g., an end face 22 as depicted in FIG. 2). In some cases, the end face may have a gas/lens wash nozzle for supplying gas to insufflate the interior of the patient at the treatment area and for supplying water to wash a lens covering the imager, an irrigation opening that may supply irrigation fluid to the treatment area (e.g., a target site) of the patient, one or more illumination windows that convey illumination light to the treatment area, and an opening to a working channel extending along the shaft 12 for passing tools to the treatment area. In some examples, the working channel may extend along the shaft 12 to a proximal channel opening 24 positioned distal to an operating handle 26 (e.g., a proximal handle) of the endoscope 10. A biopsy valve 28 may be utilized to seal the channel opening 24 against unwanted fluid egress.

The operating handle 26 may be provided with one or more knobs 30 for providing steering, such as remote 4-way steering, of the distal tip via wires connected to the articulation joint in the flexible bending portion 20 (e.g., one knob may control up-down steering and another knob may control left-right steering). A plurality of video switches 32 for remotely operating a video processing unit may be arranged on the handle 26 at the proximal portion 14 of the endoscope 10.

The handle 26 may be provided with dual valve locations 34. One of the valve locations 34 may have or receive a gas/water valve 36 for operating an insufflating gas and lens water feed operation. A gas supply line and a lens wash supply line may run distally from the gas/water valve 36 along the shaft 12 and converge at the distal tip 18 proximal to the gas/wash nozzle, but this is not required.

The other valve location 34 may have or receive a suction valve 38 for operating a suction operation. A suction supply line may run distally from the suction valve 38 along the shaft 12 to a junction point in fluid communication with the working channel of the endoscope 10.

Although an illustrative endoscope 10 is described with respect to FIG. 1, other suitable configurations of the endoscope 10 are contemplated and one or more other suitable delivery devices may be utilized with the connector concepts discussed herein. In some examples, an illustrative configuration of a delivery device may include the elongate shaft 12 and the handle 26, along with other suitable components for using the delivery device (e.g., the endoscope 10 and/or one or more other suitable delivery devices) within a subject (e.g., within the patient).

FIG. 2 depicts a schematic view of the elongate shaft 12 at the distal portion 16 of the endoscope 10. As depicted in FIG. 2, the elongate shaft 12 may include one or more working channels 40 with an opening in the end face 22. Further, the end face 22 may include openings for one or more other channels extending through the elongate shaft 12 of the endoscope 10 including, but not limited to, one or more suction/aspiration channels 42, one or more irrigation channels 44, one or more imaging channels 46, one or more light guide channels, and/or one or more other suitable channels.

In some cases, one or more elongate members 50 may extend along the elongate shaft 12 from the distal portion 16 of the endoscope 10 to the proximal portion 14 of the endoscope 10. Although not required, a distal end of the elongate member 50 may be coupled to a cap or other suitable component at a distal portion 16 of the endoscope 10 and coupled with or proximate the handle 26 at the proximal portion 14 of the endoscope 10. The elongate member(s) 50 may by any suitable type of elongate member 50 including, but not limited to coils, cabling, tubing, auxiliary working channels, aspiration channels, gas/liquid channels, light channels, optical channels, and/or other suitable types of elongate members 50.

When the elongate member(s) 50 are not constrained relative to the elongate shaft 12 (e.g., as depicted in FIG. 2), tissue may be pinched by the elongate members 50 and/or between the elongate members 50 and the elongate shaft 12 during deployment, extraction, and/or adjustment of the endoscope 10 within the patient (e.g., as a result of relative movement between the elongate member 50 and the elongate shaft 12). Even so, the elongate members 50 may extend along the elongate shaft 12 of the endoscope 10 during a procedure without a securing mechanism along the elongate shaft 12 or a sheath (e.g., a silicone sheath and/or other suitable type of sheath) may be applied over the elongate member 50 and the elongate shaft 12 to restrict relative movement thereof.

Utilizing a sheath to constrain the elongate member 50 relative to the elongate shaft 12 may add to an overall diameter of the elongate shaft 12 and the elongate member 50, which may affect a compatibility of the elongate shaft 12 with over-tubes and thus, increase a likelihood of tissue trauma and/or scraping during navigation of the elongate shaft 12 to a target site and/or extraction of the elongate shaft 12 from the patient body. Further, utilizing a sheath extending along the elongate shaft 12 may hinder a mobility of the elongate shaft 12 (e.g., may stiffen and/or reduce flexibility of the elongate shaft 12) and/or the elongate member 50, which may reduce an ability of the elongate shaft 12 to be used for navigating tortuous paths.

One or more connectors may be utilized to secure the elongate member 50 extending along the elongate shaft 12 relative to the elongate shaft 12. The connectors may extend at least partially around the elongate member 50 and the elongate shaft 12 to retain the elongate member 50 with respect to the shaft 12, while minimally increasing an overall diameter of the endoscope 10 along the elongate shaft 12, maintaining mobility of the endoscope 10 (e.g., flexibility of the elongate shaft 12 and the elongate member 50), and achieving quick and straightforward setup of the elongate member 50 relative to the elongate shaft 12. In some aspects, the connectors may allow a degree of relative movement between the elongate member and the shaft 12, such as to facilitate bending of these elements through tortuous body passages.

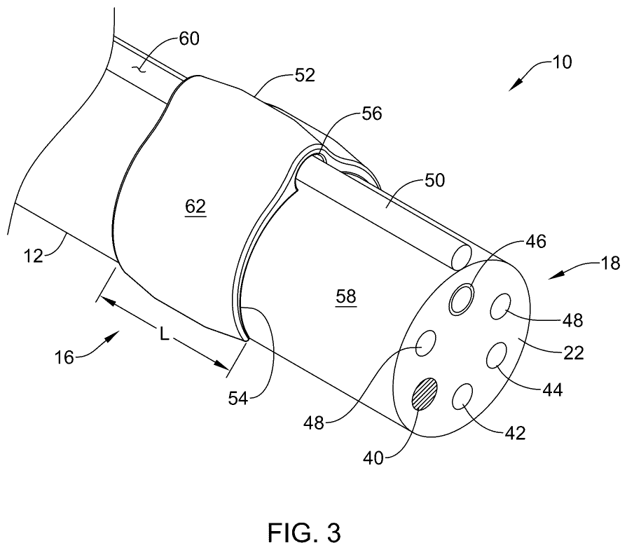

FIG. 3 depicts a schematic view of an illustrative connector 52 coupling the elongate shaft 12 and the elongate member 50. The connector 52 may be configured to extend at least partially around a circumference of the elongate shaft 12. In some cases, when the connector 52 is coupled with and/or mating with the elongate shaft 12 and the elongate member 50, the connector 52 may allow for relative longitudinal movement and/or rotational movement of the elongate member 50 relative to the elongate shaft 12 to reduce binding between elongate member 50 and the elongate shaft 12 and facilitate flexibility of the elongate shaft 12 as the endoscope 10 is maneuvered to a target site.

In some cases, the connector 52 may include one or more contoured portions or surfaces. In some examples, the connector 52 may include one or more first contoured portions 54 configured to couple or mate with the elongate shaft 12 (e.g., with an outer surface 58 of the elongate shaft 12) of the endoscope 10, and one or more second contoured portions 56 may be configured to couple or mate with the elongate member 50 (e.g., with an outer surface 60 of the elongate member 50).

When the first contoured portion 54 is mated with the outer surface 58 of the elongate shaft 12 and the second contoured portion 56 is coupled with the elongate member 50, the elongate member 50 may be secured relative to the endoscope 10 (e.g., relative to the elongate shaft 12). In some examples, when the elongate member 50 is secured relative to the endoscope 10, as discussed, there may be relative longitudinal and/or rotational movement between the elongate member 50 and the elongate shaft 12, but this is not required and elongate member 50 may be fully fixed relative to the elongate shaft 12.

The connector(s) 52 may be formed from any suitable materials. Example suitable materials from which the connector 52 may be formed include, but are not limited to, polymers, flexible polymers, metals, plastic, rubber, silicone, fabric, and/or other suitable materials. In some examples, the connector 52 may be formed with a molded plastic having a silicone or other soft polymer material on or forming the first contoured portion 54, where the molded plastic forms a lubricious or smooth surface (e.g., from a hard plastic) on an outer surface 62 of the connector 52 (e.g., a surface of the connector 52 facing a direction that faces away from a surface of the first contoured portion 54) to facilitate the connector passing along tissue. Additionally or alternatively, the molded plastic may form a lubricious or smooth surface on the second contoured portion 56 to facilitate the elongate member 50 adjusting longitudinally and/or rotationally along the second contoured portion 56. In some examples, the first contoured portion 54 may be formed from or include a lining of plastic configured to form a firm grip on the elongate shaft 12 and the outer surface 62 of the connector 52 may be formed from or include a lining of rubber or silicone to prevent mitigate tissue trauma. In some examples, the connector 52 may be monolithically formed from a flexible polymer. Other suitable configurations of materials may be utilized to form the connector 52.

The connector(s) 52 may have any suitable length L. In some examples, suitable lengths L of the connector(s) 52 are lengths L which allow flexibility of the elongate shaft 12 when the connector(s) 52 are applied thereto. For instance, the lengths L of the connector(s) 52 may be selected such that the elongate shaft 12 may maintain or substantially maintain its typical or standard flexibility, particularly relative to when a sheath is applied over the elongate shaft 12 to secure the elongate member 50 relative to the elongate shaft 12. Example suitable lengths L include, but are not limited to, lengths within a range of about five (5) millimeters (mm) to about fifty (50) mm.

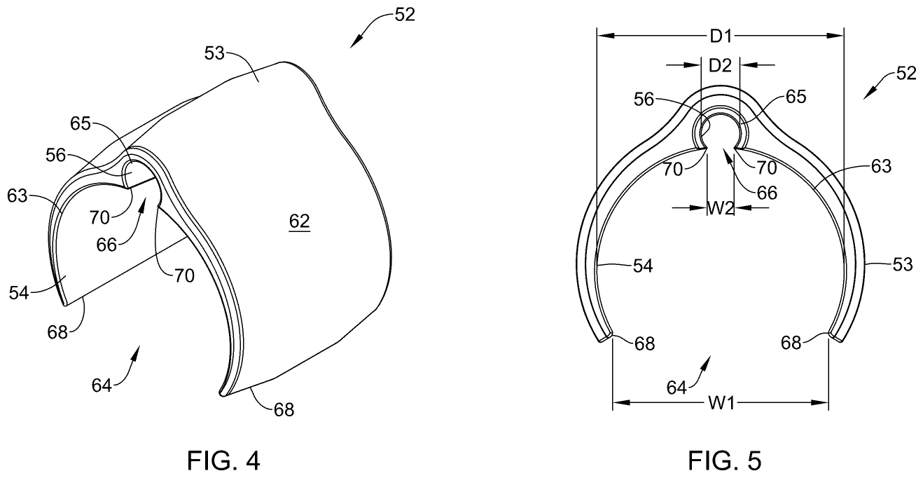

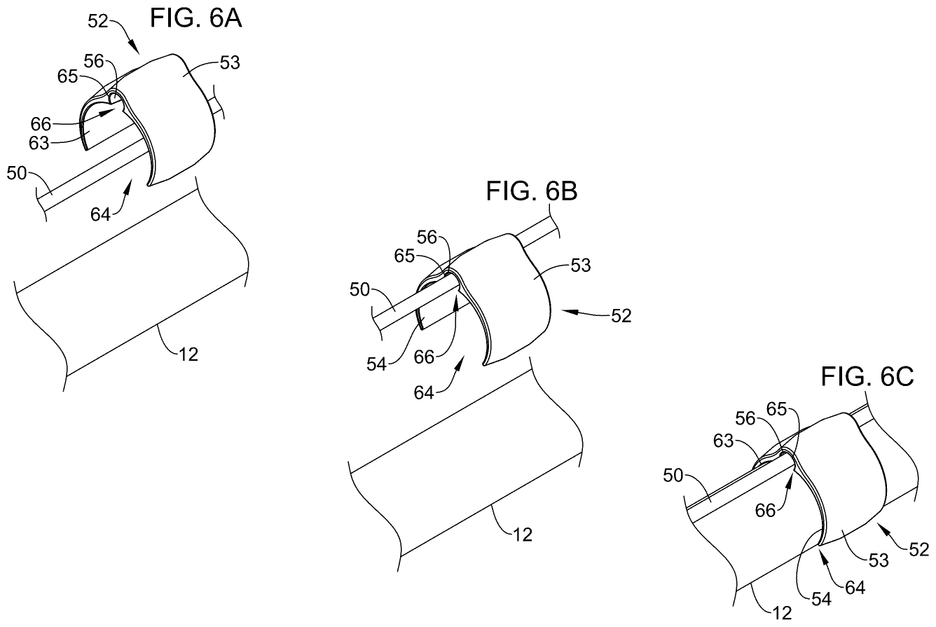

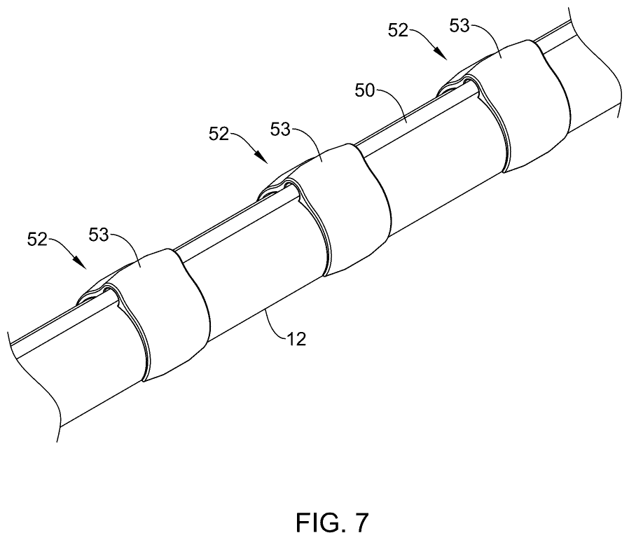

FIGS. 4-7 depict an illustrative configuration of the connector 52 configured as a fixture 53 having a clip configuration (e.g., where the connector 52 may be configured to be a clip). FIGS. 4 and 5 depict schematic perspective and end views, respectively, of the connector 52 having the clip configuration. FIGS. 6A-6C schematically depicts a technique for applying the connector 52 of FIGS. 4 and 5 to the elongate shaft 12 and the elongate member 50. FIG. 7 schematically depicts a perspective view of a plurality of the connectors 52 applied to the elongate shaft 12 and the elongate member 50.

The clip configuration of the fixture 53 may facilitate coupling the connector 52 to and the elongate member 50 tangential to the elongate shaft 12 via a snap connection and/or other suitable connection with the elongate shaft 12. Further, the clip configuration of the fixture 53 may facilitate removal of the connector 52 from the elongate shaft 12 by allowing a user to pull the fixtures 53 off of the elongate shaft 12 (e.g., unsnap the connectors 52 from the elongate shaft 12) without other adjustment of the connector 52.

The fixture 53 of the connector 52 depicted in FIG. 4 may include or define a first coupling component 63 including the first contoured portion 54 and a first opening 64 configured to receive the elongate shaft 12 (e.g., in a direction transverse to an axial direction of the elongate shaft 12). In some examples, the first opening 64 may be defined by ends 68 of the first contoured portion 54. When the material forming the first coupling component 63 is flexible and/or the outer material of the elongate shaft 12 is flexible, the elongate shaft 12 may be received in the first opening 64, snapped into the connector 52, and the first contoured portion 54 may be coupled and/or mated with the outer surface 58 of the elongate shaft 12.

The first coupling component 63 and/or the first contoured portion 54 may be configured to span greater than one hundred eighty degrees around a circumference of a received elongate shaft 12 such that the first contoured portion 54 may mate with the outer surface 58 of the elongate shaft 12 and the first coupling component 63 may engage the elongate shaft 12. Alternatively or additionally, the first contoured portion 54 and/or the first coupling component 63 may extend circumferentially within a range of one hundred eighty degrees to three hundred sixty degrees and the elongate shaft 12 may be threaded through (e.g., inserted axially through) the first contoured portion 54 of the first coupling component 63.

The fixture 53 of the connector 52 depicted in FIG. 4 may include or define a second coupling component 65 including the second contoured portion 56 and a second opening 66 configured to receive the elongate member 50. In some examples, the second opening 64 may be defined by ends 70 of the second contoured portion 56. When the material forming the second coupling component 65 is flexible and/or outer material of the elongate member 50 is flexible, the elongate member 50 may be received in the second opening 66, snapped into the connector 52, and the second contoured portion 56 may be coupled and/or mated with the outer surface 60 of the elongate member 50.

The second coupling component 65 and/or the second contoured portion 56 may be configured to span less than or greater than one hundred eight degrees around a circumference of a received elongate member 50 such that the second contoured portion 56 may mate and/or couple with the outer surface 60 of the elongate member 50. Alternatively or additionally, the second contoured portion 56 and/or the second coupling component 65 may extend circumferentially within a range one hundred eighty degrees to three hundred sixty degrees and the elongate member 50 may be threaded through (e.g., inserted axially through) the second contoured portion 56 of the second coupling component 65.

The fixture 53 including and/or defining the first coupling component 63 with the first contoured portion 54 and the second coupling component 65 with the second contoured portion 56 may be formed from one or more materials. In some examples and as depicted in FIG. 4, the first coupling component 63 and/or the first contoured portion 54 may be monolithic with the second coupling component 65 and/or the second contoured portion 56. In some cases, when monolithically formed, one or more surface treatments and/or surface materials (e.g., silicone, etc.) may be applied to a monolithic material of the fixture 53 to create a desired surface texture and/or achieve a desired surface parameter or configuration.

The first opening 64 of the connector 52, when included, may have any suitable width W1. In some examples, the width W1 may be any suitable size for receiving the elongate shaft 12 therethrough when snapping the first coupling component 63 onto the elongate shaft 12 and preventing or mitigating unintentional disengagement between the first coupling component 63 and the elongate shaft 12. Example suitable widths W1 include, but are not limited to, widths within a range of about one (1) mm to about twenty (20) mm.

The first coupling component 63 and the first contoured portion 54 may have an internal diameter D1 of any suitable size. In some examples, the diameter D1 may be any suitable size for receiving, coupling, and/or mating with the elongate shaft 12. In some examples, the internal diameter D1 may be sized to create a close fit with an outer diameter of the elongate shaft 12 to prevent or mitigate movement of the connector 52 about the elongate shaft 12 and maintain a desired diameter of the endoscope 10 at the connector 52 that may mitigate tissue trauma proximate the elongate shaft 12 during navigation to a target site. Example suitable internal diameters D1 include, but are not limited to, internal diameters within a range of about five (5) mm to about twenty (20) mm.

The second opening 66 of the connector 52, when included, may have any suitable width W2. In some examples, the width W2 may be any suitable size for receiving the elongate member 50 therethrough when engaging the elongate member 50 at or in the second coupling component 65 and preventing or mitigating unintentional disengagement between the second coupling component 65 and the elongate member 50. Example suitable widths W2 include, but are not limited to, widths within a range of about 0.1 mm to about five (5) mm.

The second coupling component 65 and the second contoured portion 56 thereof may have an internal diameter D2 of any suitable size. In some examples, the internal diameter D2 may be any suitable size for receiving, coupling, and/or mating with the elongate member 50. In some examples, the internal diameter D2 may be sized to create a loose fit with an outer diameter of the elongate member 50 to allow the elongate member 50 to slide or otherwise adjust (e.g., longitudinally and/or rotationally slide) relative to the connector 52 and/or the elongate shaft 12 when the connector 52 is coupled with the elongate shaft 12. Example suitable internal diameters D2 include, but are not limited to, internal diameters within a range of about 0.2 mm to about ten (10) mm.

As discussed, FIGS. 6A-6C depict an illustrative technique for applying the connector 52 having the clip configuration, the first opening 64, and the second opening 66 to the elongate member 50 and the elongate shaft 12. Although the connectors 52 may be provided pre-coupled with the elongate member 50 in a package, the connector(s) 52 and the elongate member 50 may be provided separate from one another, as depicted in FIG. 6A. When provided separate from one another and the second coupling component 65 includes the second opening 66, the second opening 66 may be aligned with the elongate member 50 for snapping or otherwise inserting the elongate member 50 into the second coupling component 65, as depicted in FIG. 6A. Alternatively, with or without the second opening 66, the elongate member 50 may be aligned with the second coupling component 65 to be threaded axially into and/or through the second coupling component 65.

As depicted in FIG. 6B, the elongate member 50 may be engaged with (e.g., mated with, snapped into, threaded through, coupled with, etc.) the second coupling component 65 and the second contoured portion 56 through the second opening 66. Once the elongate member 50 has been coupled with the second contoured portion 56 and/or the second coupling component 65, the first opening 64 may be aligned with a desired axial location between a first end and a second end of the elongate shaft 12 at which the connector 52 is to be mated and/or coupled with the elongate shaft 12.

As depicted in FIG. 6C, the elongate shaft 12 may be engaged with (e.g., mated with, snapped into, coupled with, threaded through, etc.) the first coupling component 63 and the first contoured portion 54 at the desired axial location along the elongate shaft 12. In some examples, the first coupling component 63 may have some flexibility or bend and may be configured to adjust radially outward to receive the elongate shaft 12 through the first opening 64 with a snap connection, but this is not required.

In some cases, a plurality of connectors 52 may be utilized to secure the elongate member 50 relative to the elongate shaft 12, as depicted in FIG. 7. When a plurality of connectors 52 are utilized to secure the elongate member 50 relative to the elongate shaft 12, the connectors 52 may be spaced from one another at equal or different intervals. In some examples, the plurality of connectors 52 may be spaced apart by a desired interval (e.g., by a desired distance, different distances, same distances, etc.) so as to maintain a desired flexibility along a length of the elongate shaft 12 of the endoscope 10.

The connectors 52 may be individually applied to the elongate shaft 12 at the desired interval and/or applied in one or more other suitable manners. The connectors 52 may be removed from the elongate shaft 12 by individually pulling the connectors 52 off of the elongate shaft 12 such that the connectors 52 may remain engaged with the elongate member 50, but this is not required. Alternatively or additionally, a user may pull on the elongate member 50 which may unsnap or otherwise remove the connectors 52 from the elongate shaft 12 while maintaining the connectors 52 on or otherwise engaged with the elongate member 50.

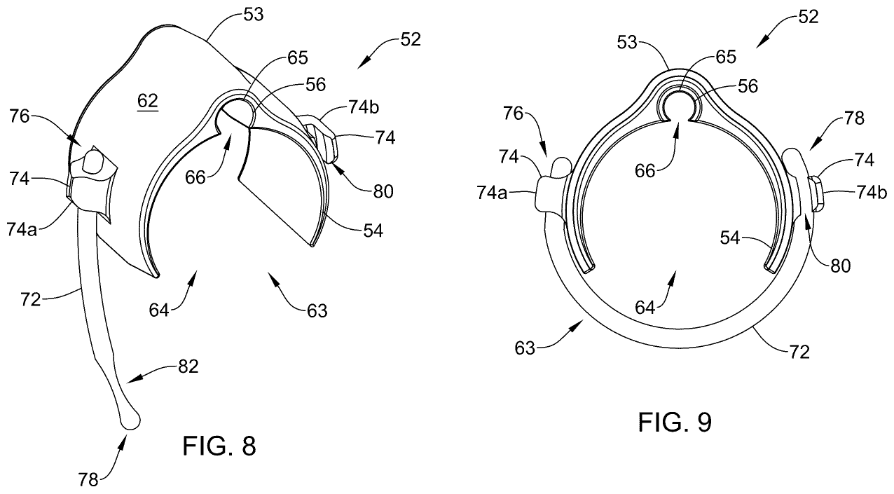

FIG. 8 depicts a schematic perspective view of a connector 52 that comprises a configuration of the fixture 53 and a configuration of a band 72 (e.g., a strap and/or other suitable band). The configuration of the fixture 53 depicted in FIG. 8 may be similar to the configuration of the fixture 53 depicted in FIGS. 4-7, but with engagement components 74 (e.g., holders, tabs, clips, etc.) extending laterally from the outer surface 62. However, other suitable configurations of the fixture 53 that may be utilized with the band 72 are contemplated.

In the configuration of the connector 52 depicted in FIG. 8, the fixture 53 may include at least a first engagement component 74a and a second engagement component 74b. In some examples, the first engagement component 74a may be configured to engage a first end 76 of the band 72 and the second engagement component 74b may be configured to engage a second end 78 of the band 72.

The first engagement component 74a may be configured to fixedly engage the first end 76 of the band 72 in any suitable manner. In some examples, the first engagement component 74a may engage the first end 76 of the band 72 with a friction fit, a pinch connection, a tie mechanism, a clip, and/or with one or more other suitable connection types. In one example, the first engagement component 74a may be configured to fixedly engage the first end 76 of the band 72 via a friction fit and may include a lumen or opening extending through the first engagement component 74a. When the first end 76 of the band 72 is to be fixedly coupled with the first engagement component 74a, the first end 76 of the band 72 may be axially or longitudinally inserted through or threaded into an opening in the first engagement component 74a until the band 72 is secured in the first engagement component 74a.

The second engagement component 74b may be configured to releasably engage the second end 78 of the band 72 in any suitable manner. In some examples, the second engagement component 74b may engage the first end 76 of the band 72 with a friction fit, a pinch connection, a tie mechanism, a clip, and/or with one or more other suitable connections. In one example, the second engagement component 74b may be configured to releasably engage the second end 78 of the band 72 via friction fit or a pinch or clip fit. The second engagement component 74b may have a clip or tab configuration with an opening 80 configured to receive and engage a lateral side of the band 72 at or proximate the second end 78, but other suitable configurations are contemplated.

The engagement components 74 may be positioned at any suitable axial and/or circumferential locations along the fixture 53. In some examples, the first engagement component 74a and the second engagement component 74b may be located at a same axial location along the fixture 53, but this is not required and the engagement components 74 may be located at different axial locations. In some examples, the first engagement component 74a and the second engagement component 74b may be located at the same axial location along the fixture 53 and may be spaced one hundred eight degrees apart or less along the fixture, but other suitable configurations are contemplated.

When the second end 78 of the band 72 is to be releasably coupled with the second engagement component 74b, the second end 78 of the band 72 and/or a portion of the band 72 proximate the second end 78 may be laterally and/or otherwise inserted through the opening 80 and pulled axially or longitudinally until the band 72 is releasably secured with the second engagement component 74b in a manner that is configured to secure the connector 52 at a longitudinal or axial location along the elongate shaft 12. In some examples, the second end 78 of the band 72 may be disengaged from the second engagement component 74b by pulling on the band 72 in a lateral direction through the opening 80.

The band 72 may be made from any suitable material. Suitable materials for the band 72 may include, but are not limited to, polymers, metals, elastic materials, fabric materials, braided materials, rubber, silicone, and/or other suitable materials. In some examples, the band 72 may be formed from an elastic polymer material, but this is not required. When formed with an elastic material, the band 72 may be stretched as the band 72 is being coupled to the second engagement component 74b so as to engage the elongate shaft 12 to secure the connector 52 at a desired longitudinal or axial location along the elongate shaft 12.

The band 72 may include one or more engaging locations 82, which may facilitate the band engaging the one or more of the engagement components 74. As depicted in FIG. 8, the band 72 may include an engaging location 82 at least at or proximate the second end 78 that may facilitate the band engaging the second engagement component 74b, but this is not required. When included in the band 72, the engaging location 82 may be a protrusion, an indent (e.g., as depicted in FIG. 8), and/or other suitable features configured to facilitate the band 72 engaging the second engagement component 74b.

FIG. 9 depicts a schematic end view of the illustrative configuration of the connector 52 depicted in FIG. 8. The second end 78 of the band 72 may engage the second engagement component 74b to close off the first opening 64, as depicted in FIG. 9. When the elongate shaft 12 is mated with the first contoured portion 54, the band 72 releasably secured with the second engagement component 74b may facilitate maintaining the connector 52 at a desired longitudinal or axial location and/or circumferential location along the elongate shaft 12.

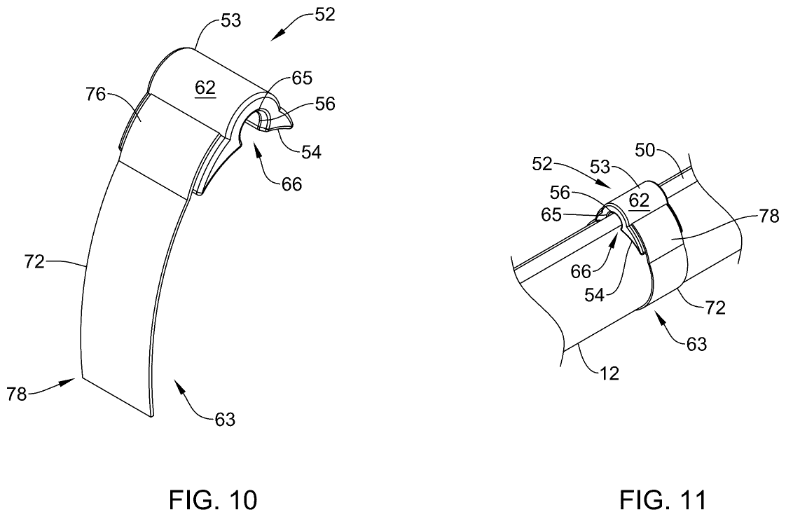

FIG. 10 depicts a schematic perspective view of a connector 52 that comprises a configuration of the fixture 53 and a configuration of the band 72 (e.g., a strap and/or other suitable band) configured to wrap around an outer surface or diameter of the elongate shaft 12. The configuration of the fixture 53 depicted in FIG. 10 may include the first contoured portion 54 of the first coupling component 63 configured to mate with the elongate shaft 12 and the second contoured portion 56 of the second coupling component 65 configured to mate and/or couple with the elongate member 50. The second coupling component 65 in the configuration of the connector 52 depicted in FIG. 10 may be similar to the second coupling component 65 discussed with respect to FIGS. 4-7, however, other suitable configurations are contemplated.

The first coupling component 63 may include the first contoured portion 54 defined by the fixture 53 and the band 72. The first end 76 of the band 72 may be fixedly secured to the fixture 53, as depicted in FIG. 10, and the second end 78 of the band 72 may be configured to be releasably secured to the fixture 53 via an adhesive and/or other suitable material. Although not required, the first end 76 and/or the second end 78 of the band 72 may be secured to the outer surface 62 of the fixture 53.

The band 72 in the configuration of the connector 52 depicted in FIG. 10 may have any suitable configuration. In some cases, the shape, size, and/or material used for the band 72 may be selected based on a size and/or configuration of the elongate shaft 12 and/or based on other suitable parameters. In some examples, the band 72 may be formed from a thin strip of adhesive, as depicted in FIG. 10, but this is not required. Example suitable adhesive materials include, but are not limited to, acrylic adhesive, tape, self-fusing silicone, pressure activated adhesive, glue, and/or other suitable adhesive materials. Although the band 72 may be configured to stick to the elongate shaft 12 and/or fixture 53 (e.g., the outer surface 62 of the fixture 53), the first end 76 and/or the second end 78 of the band 72 may be secured to the fixture 53 using an adhesive (e.g., glue, self-fusing silicone, etc.)

FIG. 11 depicts a schematic end view of the illustrative configuration of the connector 52 depicted in FIG. 10. As depicted in FIG. 10, the band 72 may be wrapped around an outer diameter of the elongate shaft 12, around the fixture 53 (e.g., the outer surface 62 of the fixture 53), and the second end 78 of the band 72 may engage the fixture 53 to secure the elongate shaft 12 within the first coupling component 63 and the elongate member 50 within the second coupling component 65 and relative to the elongate shaft 12. When the elongate shaft 12 is mated with the first contoured portion 54, the band 72 releasably secured with the second engagement component 74b may facilitate maintaining the connector 52 at a desired longitudinal or axial location and/or circumferential location along the elongate shaft 12 via a pressure or friction connection, an adhesive connection, and/or other suitable connection.

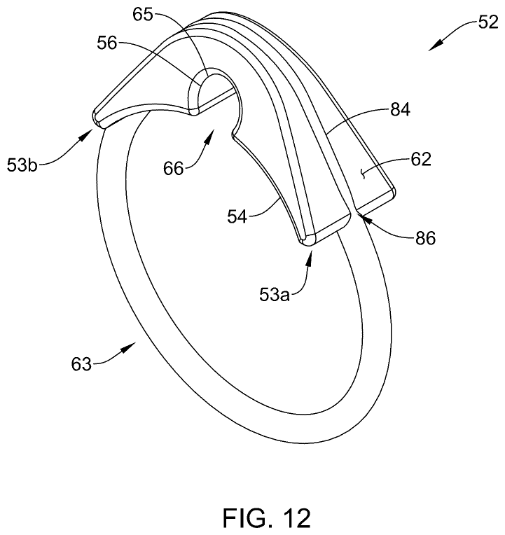

In an additional or alternative configuration of the connector 52, the first coupling component 63 may include the first contoured portion 54 and a configuration of the band 72 taking on an o-ring configuration that may be designed to fit around the elongate shaft 12 (e.g., fit around an outer surface 62 of the elongate shaft 12) of the endoscope 10. FIG. 12 depicts a schematic perspective view of the connector 52 with an illustrative fixture 53 configured to receive an o-ring configuration of the band 72.

The configuration of the fixture 53 depicted in FIG. 12 may be similar to the configuration of the fixture 53 depicted in FIGS. 10 and 11, but with a groove 84 extending inward from the outer surface 62 of the fixture 53 and configured to receive the band 72. However, other suitable configurations of the fixture 53 that may be utilized with the band 72 are contemplated.

The first coupling component 63 may include the first contoured portion 54 defined by the fixture 53 and the band 72. As discussed, the band 72 may have an o-ring configuration and/or other suitable configuration designed to engage the fixture 53 and the elongate shaft 12. In some examples, the band 72 may be configured to engage the groove 84 extending along the outer surface 62 of the fixture 53.

The groove 84 extending into the fixture 53 from the outer surface 62 of the fixture 53 may be configured to receive and engage the band 72 to secure the band 72 relative to the fixture 53. The groove 84 may be configured to extend from a first end 53a of the fixture 53 to a second end 53b of the fixture 53, but this is not required. In some cases, the groove 84 may be configured to receive an entirety of (e.g., as depicted in FIG. 12) or at least part of a circumference of the band 72 in an axial direction. The groove 84 may be a press-fit groove and/or other suitable type of groove.

The groove 84 may have an opening 86 therealong that has a width configured to receive a compressible portion of a band 72 when in a compressed configuration, where the band 72 may be in an uncompressed or partially compressed configuration once inserted into the groove 84 past the opening 86, but this is not required. A width of the opening 86 may have any suitable size and configuration. In some examples, the width of the opening 86 may have a width that spans a distance that is less than a distance of a diameter (e.g., a maximum width) of the groove 84, equal to the distance of the diameter of the groove 84, greater than the distance of the diameter of the groove, and/or other suitable size relative to a maximum width of the groove 84. In some examples, a distance of the width of the opening 86 may vary along a length of the opening 86 or may be constant along the length of the opening 86.

The groove 84 may be positioned at any suitable axial and/or circumferential locations along the fixture 53. In some examples, the groove 84 may be located at a constant axial location along the fixture 53, but this is not required, and the groove 84 may be located at different suitable axial locations along the fixture 53.

The band 72 having an o-ring configuration may be made from any suitable material. Suitable materials for the band 72 having the o-ring configuration may include, but are not limited to, polymers, metals, elastic materials, fabric materials, braided materials, rubber, silicone, and/or other suitable materials. In some examples, the band 72 may be formed from an elastic polymer material, but this is not required. When formed with an elastic material, the band 72 having the o-ring configuration may be stretched as the band 72 is being coupled to the fixture 53 (e.g., in the groove 84) and the elongate shaft 12 to secure the connector 52 at a desired longitudinal or axial location along the elongate shaft 12.

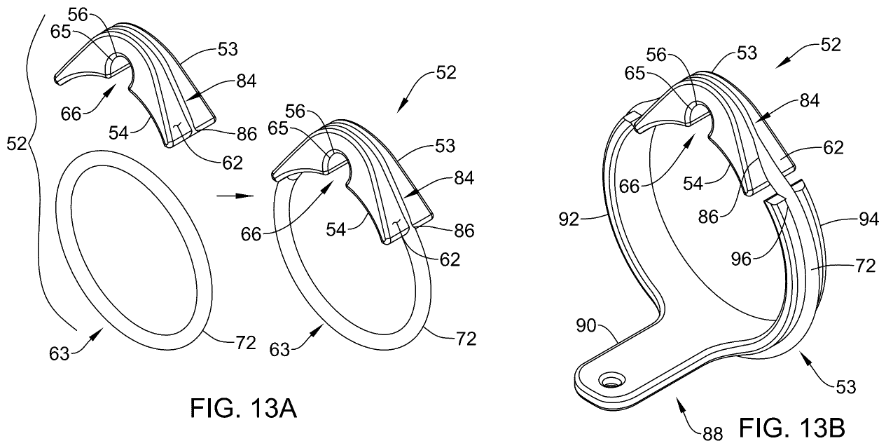

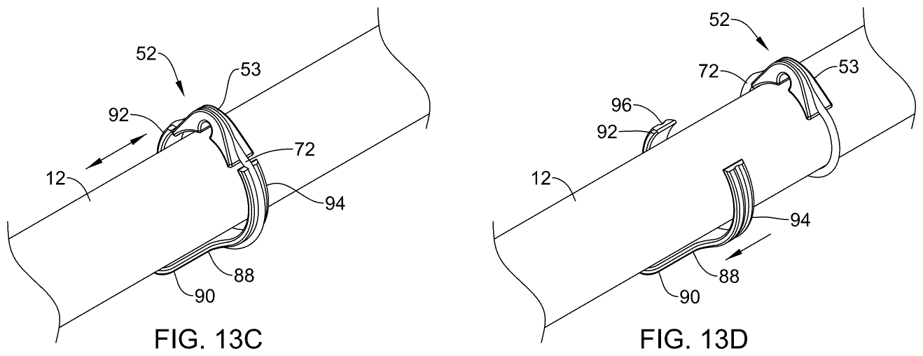

FIGS. 13A-D depict an illustrative technique for applying the connector 52 including the fixture 53 and the band 72 in an o-ring configuration to the elongate member 50 and the elongate shaft 12. Although the connectors 52 may be provided pre-coupled with the elongate member 50 in a package, the connector(s) 52 and the elongate member 50 may be separate from one another. Illustration of coupling of the elongate member 50 with the fixture 53 before coupling the connector 52 to the elongate member 50 has been omitted from FIGS. 13A-13D.

FIG. 13A depicts, on the left, the connector 52 with the fixture 53 and the band 72 separate from one another (e.g., in a disengaged configuration). On the right, FIG. 13A depicts the connector 52 with the fixture 53 and the band 72 connected (e.g., in an engaged configuration). When connected (e.g., in the engaged configuration), the band 72 may be in the groove 84 (e.g., the band 72 may have been inserted through the opening). In some examples, the connector 52 may be provided in a package with the band 72 pre-engaged with the groove 84 of the fixture, but this is not required.

Optionally, a tool 88 (e.g., a delivery tool) may be utilized to position the connector 52 at and/or to apply the connector 52 to the elongate shaft 12. As depicted in

FIG. 13B, the tool 88 may be inserted within a circumference of the band 72 and the fixture 53 to expand an inner diameter of the band 72. In some examples, the tool 88 may be configured to expand the band 72 of the first coupling component 63 to a diameter greater than a diameter (e.g., an outer diameter) of the elongate shaft 12 for delivery of the first coupling component 63 and the second coupling component 65 of the connector 52 to a position along a length of the elongate shaft 12.

The tool 88 may have any suitable configuration. In some examples, the tool 88 may include a handle 90, a first arm 92 extending outward from the handle 90 in a first direction and a second arm 94 extending outward from the handle in a second direction. The first arm 92 and the second arm 94 may be rounded so as to be configured to engage the band 72 in the circular o-ring configuration. Further, ends of the first arm 92 and the second arm 94 may be circumferentially spaced from one another (e.g., as depicted in FIG. 13B) or may touch to form a closed circle. The first arm 92 and the second arm 94 may be spaced from one another so as to form an inner diameter with or without the connector 52 that may be greater than at least a portion of an outer diameter of the elongate shaft 12 and greater than the band 72 in a resting or relaxed, non-expanded configuration.

An outer surface of the first arm 92 and/or the second arm 94 may include one or more grooves 96. The groove 96, when included, may be configured to receive the band 72 to facilitate maintaining the band at a desired location relative to (e.g., along the length of) the tool 88 and/or for other suitable purposes.

FIG. 13C depicts positioning (e.g., two-way adjustable positioning represented by the double arrow) the connector 52 with the band 72 in the o-ring configuration around the elongate shaft 12 using the tool 88. As depicted in FIG. 13C, the first arm 92 and the second arm 94 may form, with the fixture 53, an inner diameter greater than an outer diameter of the elongate shaft 12 such that the connector may be positioned at a desired location (e.g., a longitudinal and/or circumferential location) along the elongate shaft 12.

Once at a desired location along the elongate shaft 12, the connector 52 may be separated from the tool 88, as depicted for example in FIG. 13D (e.g., the tool 88 may be withdrawn axially from the connector 52 and/or separated in one or more other suitable manners), and the band 72 may adjust to a less expanded state or configuration that is configured to facilitate securing the connector to the elongate shaft 12. The connector 52 may be separated from the tool 88 in any suitable manner. In some examples, the band 72 of the connector 52 may be rolled and/or slid out of the groove 96 and onto the elongate shaft 12 to separate the connector 52 from the tool 88. Other suitable separation techniques are contemplated.

Once separated, the connector 52 may be applied to the elongate shaft 12 and may fit snugly over the elongate shaft 12 so as to be secured relative to a desired location along the elongate shaft 12. Further, the tool 88 may be withdrawn from around the elongate shaft 12 once disengaged from the connector 52, as depicted in FIG. 13D. Although the tool 88 is depicted as being withdrawn in the direction of the illustrated directional arrow, the tool 88 may be withdrawn in one or more additionally or alternative directions.

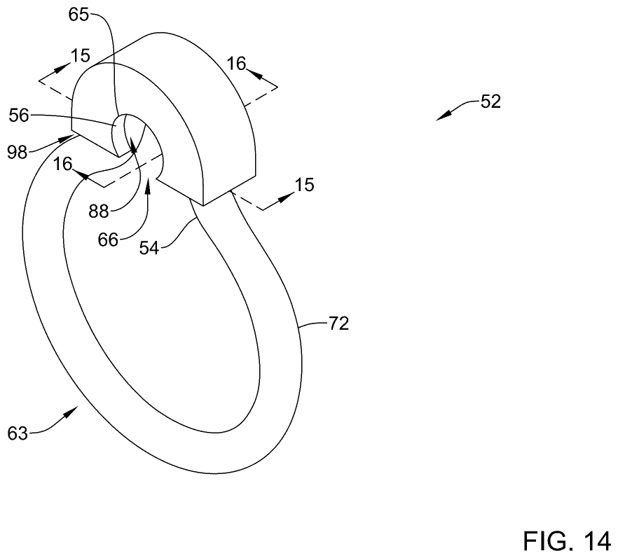

FIG. 14 depicts a schematic perspective view of an illustrative configuration of the connector 52, in which the groove 84 may extend into the fixture 53 from a bottom 98 of the fixture 53. With the groove 84 extending into the bottom 98 of the fixture 53, the band 72 may entirely form and/or substantially define the first coupling component 63 and the first contoured portion 54 thereof. The band 72 may be similar to the band 72 described above with respect to FIG. 12, but other suitable configurations are contemplated. The second coupling component 65 in the configuration of the connector 52 depicted in FIG. 14 may be similar to the second coupling component 65 discussed with respect to FIGS. 4-7, however, other suitable configurations are contemplated.

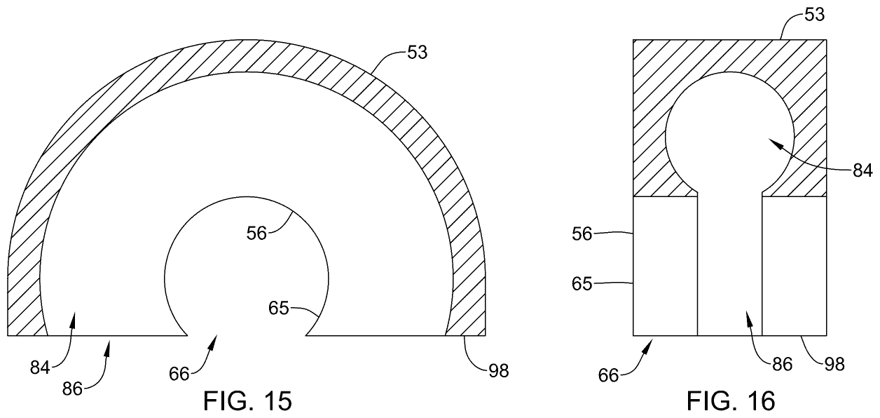

FIG. 15 depicts a schematic cross-section view of the illustrative fixture 53 configuration depicted in FIG. 14, taken along line 15-15. FIG. 16 depicts a schematic cross-section view of the illustrative fixture 53 configuration depicted in FIG. 14, taken along line 16-16. To engage the band 72 with the fixture 53, the band 72 may be press fit or snap fit into the opening 86 and into the groove 84, similar to as discussed with respect to the groove 84 being positioned through the outer surface 62 of the fixture 53.

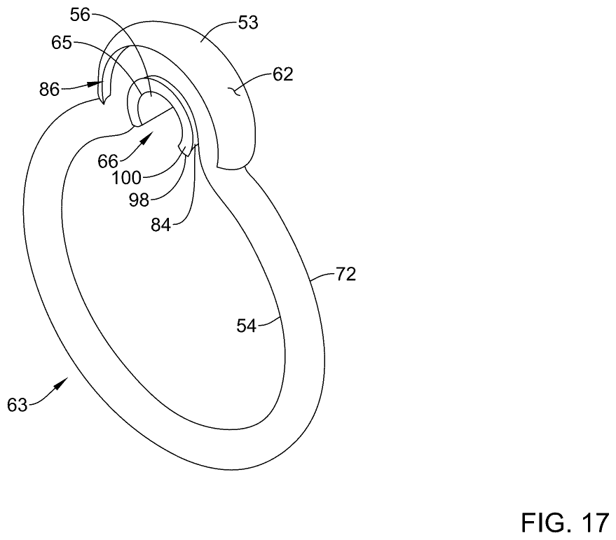

FIG. 17 depicts a schematic perspective view of an illustrative configuration of the connector 52, where the groove 84 may extend into the fixture 53 from a side 100 of the fixture 53 between the bottom 98 and the outer surface 62. With the groove 84 extending into the side 100 of the fixture 53, the band 72 may entirely form and/or substantially define the first coupling component 63 and the first contoured portion 54 thereof. The band 72 may be similar to the band 72 described above with respect to FIG. 12, but other suitable configurations are contemplated. The second coupling component 65 in the configuration of the connector 52 depicted in FIG. 17 may be similar to the second coupling component 65 discussed with respect to FIGS. 4-7, however, other suitable configurations are contemplated.

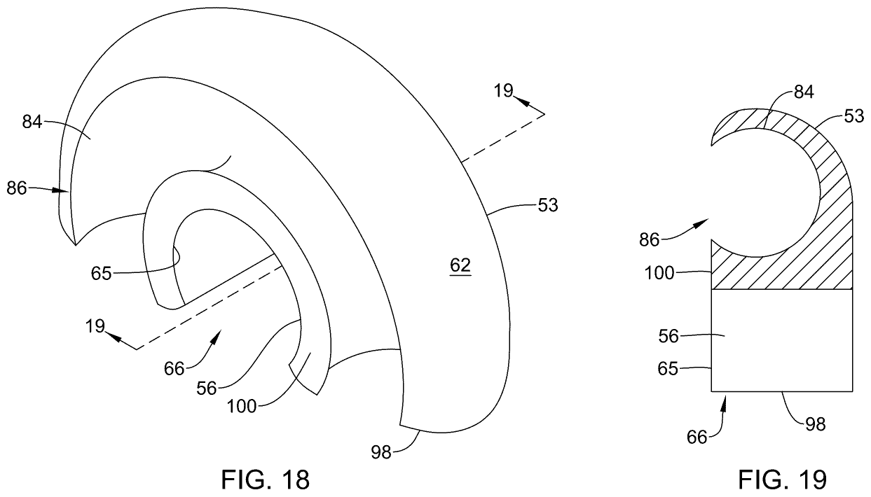

FIG. 18 depicts a schematic perspective view of the illustrative configuration of the fixture 53 depicted in FIG. 17. As discussed, the groove 84 configured for receiving the band 72 may extend into the fixture 53 from the opening 86 in the side 100 of the fixture 53.

FIG. 19 depicts a schematic cross-section view of the illustrative fixture 53 configuration depicted in FIG. 18, taken along line 19-19. To engage the band 72 with the fixture 53, the band 72 may be press fit or snap fit into the opening 86 and into the groove 84, similar to as discussed with respect to the groove 84 being positioned through the outer surface 62 of the fixture 53 and/or the groove 84 being positioned through the bottom 98 of the fixture 53.

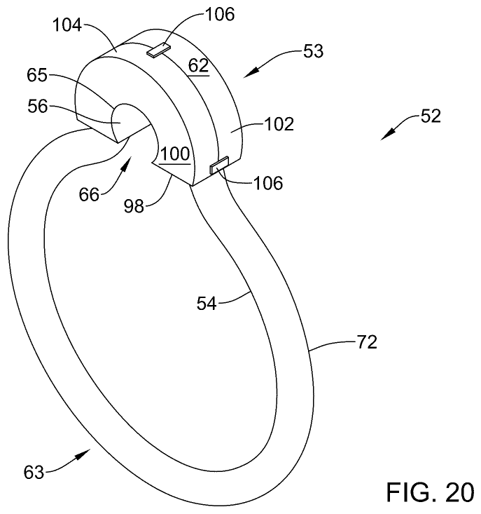

FIG. 20 depicts a schematic perspective view of an illustrative configuration of the connector 52, where the groove 84 may extend into the fixture 53 from the bottom 98 of the fixture 53 and fixture 53 may have separable components with each component defining a portion of the groove 84. With the groove 84 extending into the bottom 98 of the fixture 53, the band 72 may entirely form and/or substantially define the first coupling component 63 and the first contoured portion 54 thereof. The band 72 may be similar to the band 72 described above with respect to FIG. 12, but other suitable configurations are contemplated. The second coupling component 65 in the configuration of the connector 52 depicted in FIG. 20 may be similar to the second coupling component 65 discussed with respect to FIGS. 4-7, however, other suitable configurations are contemplated.

The fixture 53 having separable components may include any suitable number of separable components. In some examples, the fixture 53 may have a first separable component 102 (e.g., a first sub-component) and a second separable component 104 (e.g., a second sub-component) defining a compartment including the groove 84 and configured to receive the band 72, where the first separable component 102 and the second separable component 104 may be configured to engage one another.

The first separable component 102 and the second separable component 104 may be configured to engage one another in any suitable manner. Example suitable engaging configurations may include, but are not limited to, snap connections, latch connections, friction fit connections, and/or other suitable engaging techniques. In some examples, one or both of the first separable component 102 and the second separable component 104 may include one or more latches 106 configured to create a snap-fit connection with the other of the first separable component 102 and the second separable component 104.

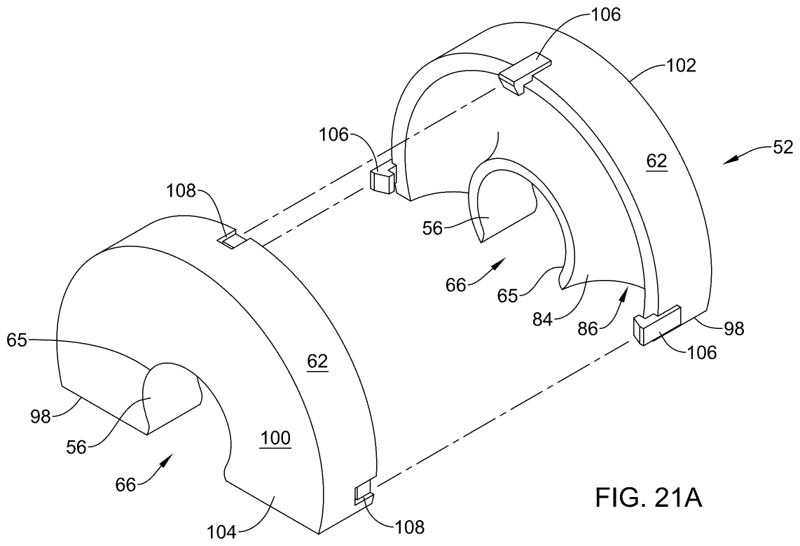

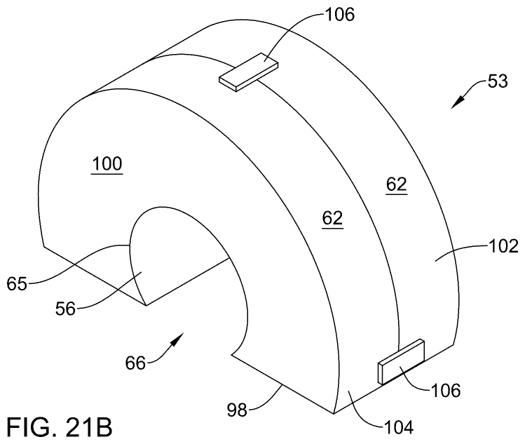

FIGS. 21A and 21B depict an illustrative technique for coupling the first separable component 102 with the second separable component 104 of the fixture 53. FIG. 21A depicts the first separable component 102 separated from the second separable component 104 of the fixture 53. FIG. 21B depicts the first separable component 102 coupled or engaged with the second separable component 104 of the fixture 53.

As depicted in FIG. 21A the first separable component 102 may define at least part of the groove 84 and the opening 86. In some case, but not required, the second separable component 104 may define at least part of the groove 84 and the opening 86 in a similar manner as the first separable component 102. Alternatively, one of the first separable component 102 and the second separable component 104 may substantially or entirely define the groove 84 and the other of the first separable component 102 and the second separable component 104 may define a cover for the groove 84 (e.g., without defining a portion of the groove).

The band 72 may be inserted into the groove 84 when the first separable component 102 and the second separable component 104 are separated from one another (e.g., in a configuration similar to as depicted in FIG. 21A. Once the band 72 is inserted into the groove 84 of one or both of the first separable component 102 and the second separable component 104, the first separable component 102 and the second separable component 104 may be brought together and the latch(es) 106 may engage respective latch engagement features 108 with a snap-fit connection and/or other suitable connection. FIG. 21B depicts the first separable component 102 engaging the second separable component 104 with the latches 106.

The fixture 53 may include any suitable number of latches 106 and latch engagement feature 108. In some examples, the fixture 53 may include three latches 106 and three respective latch engagement features 108, but this is not required and other suitable number of latches 106 and/or latch engagement features 108 may be utilized.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A device for coupling an elongate member with a shaft of an endoscope, the device comprising:
a first contoured portion configured to mate with an outer surface of the shaft of the endoscope; and
a second contoured portion configured to couple with the elongate member, and
wherein when the first contoured portion is mated with the outer surface of the shaft and the second contoured portion is coupled with the elongate member, the elongate member is secured relative to the shaft and is adjustable relative to the shaft.

2. The device of claim 1, wherein the second contoured portion is configured such that the elongate member is able to adjust rotationally and longitudinally relative to the shaft when the elongate member is coupled with the second contoured portion and the first contoured portion is mated with the outer surface of the shaft.

3. The device of claim 1, wherein the second contoured portion is part of a coupling component configured to couple with the elongate member via a snap connection.

4. The device of claim 1, wherein the first contoured portion is part of a coupling component configured to couple with the shaft via a snap connection.

5. The device of claim 1, wherein the first contoured portion and the second contoured portion are monolithic.

6. The device of claim 1, further comprising:
a fixture defining the first contoured portion and the second contoured portion; and
a band configured to engage the fixture and couple the fixture to the shaft of the endoscope.

7. The device of claim 6, wherein the band is configured to extend around the shaft and an outer surface of the fixture, the outer surface of the fixture faces a direction that is facing away from a surface of the first contoured portion.

8. The device of claim 6, wherein the band has a first end secured to the fixture and a second end secured to the fixture.

9. The device of claim 1, further comprising:
a band defining the first contoured portion; and
a fixture configured to secure the band relative to the shaft of the endoscope, wherein the fixture defines the second contoured portion and a compartment configured to receive the band.

10. The device of claim 9, wherein the fixture includes a first sub-component and a second sub-component configured to couple with the first sub-component to define the compartment.

11. The device of claim 1, further comprising:
a band defining the first contoured portion; and
a fixture comprising an opening configured to receive the band, and
wherein the opening is located at one of a bottom of the fixture configured to face the shaft of the endoscope and a side of the fixture configured to face a direction parallel to a longitudinal axis of the shaft when the band is received within the fixture and the first contoured portion is mated with the shaft.

12. A system for coupling an elongate member with a shaft of an endoscope, the system comprising:
a first coupling component; and
a second coupling component configured to couple with the elongate member, and
wherein:
the first coupling component is adjustable relative to the second coupling component to releasably couple with the shaft of the endoscope, and
when the first coupling component is releasably coupled with the shaft and the second coupling component is coupled with the elongate member, the elongate member is secured relative to the endoscope and is able to move longitudinally relative to the shaft.

13. The system of claim 12, wherein the first coupling component is annular and flexible, and the system further comprises:
a delivery tool configured to expand the first coupling component to a diameter greater than a diameter of the shaft for delivery of the first coupling component and the second coupling component to a position along a length of the shaft.

14. The system of claim 13, wherein the second coupling component has a groove in an outer surface, the groove is configured to receive the first coupling component.

15. The system of claim 13, wherein the delivery tool has a groove in an outer surface, the groove is configured to receive the first coupling component.

16. The system of claim 12, wherein the first coupling component is a portion of a clip and the second coupling component is a portion of the clip.

17. A method of coupling an elongate member to a shaft of an endoscope, the method comprising:
coupling a first coupling component to the shaft of the endoscope, wherein the elongate member is coupled to a second coupling component and coupling the first coupling component to the shaft secures the elongate member relative to the shaft at an axial location between a first end of the shaft and a second end of the shaft, and
wherein the elongate member is able to move longitudinally relative to the second coupling component when coupled with the second coupling component and the first coupling component is coupled with the shaft.

18. The method of claim 17, further comprising:
coupling the second coupling component with the elongate member, and
wherein coupling the second coupling component with the elongate member includes snapping the elongate member into the second coupling component.

19. The method of claim 17, wherein the first coupling component is a portion of a clip and the second coupling component is a portion of the clip.

20. The method of claim 17, wherein the first coupling component comprises a band and coupling the first coupling component to the shaft comprises positioning the band around the shaft.
